(19) United States
(12) Patent Application Publication
(10) Pub. No.: US 2016/0311760 A1
(43) Pub. Date: Oct. 27, 2016

(54) COMPOUNDS FOR TREATMENT OF CARDIAC ARRHYTHMIAS

(71) Applicants: ELEX BIOTECH, LLC, Portland, OR (US); BAYLOR COLLEGE OF MEDICINE, Houston, TX (US)

(72) Inventors: Robert M. Strongin, Portland, OR (US); Jonathan J. Abramson, Portland, OR (US); Martha Sibrian-Vazquez, Portland, OR (US); Xander Wehrens, Houston, TX (US)

(21) Appl. No.: 15/102,266

(22) PCT Filed: Dec. 5, 2014
(86) PCT No.: PCT/US2014/068917

§ 371 Date: Jun. 6, 2016

Related U.S. Application Data
(60) Provisional application No. 61/912,333, filed on Dec. 5, 2013.

(51) Int. Cl. C07C 229/64; C07C 219/34; C07C 229/60

(52) U.S. Cl. CPC C07C 229/64; C07C 229/60; C07C 219/34

(57) ABSTRACT

Compounds and methods thereof for reducing cardiac arrhythmia are described. In particular, compounds generated by adding chemical groups that enhance the electron donor properties of RyR inhibitors may increase inhibitor potency and thus allow for new more potent anti-arrhythmic drugs. One advantage of the compounds and methods described is a potential for drugs with enhanced electron donor properties that may be used at lower concentrations and exhibit less non-specific effects.

### CROSS REFERENCE TO RELATED APPLICATIONS

This application claims priority to U.S. Provisional Patent Application No. 61/912,333, entitled “COMPOUNDS FOR TREATMENT OF CARDIAC ARRHYTHMIAS,” filed Dec. 5, 2013, the entire contents of which are hereby incorporated by reference for all purposes.

### FIELD

The present description relates to compounds and methods for modulating the activity of calcium ion channels, including Ca2+-induced (or Ca2+-activated) calcium release channels and conformationally coupled calcium release channels such as ryanodine receptors in a subject.

### BACKGROUND AND SUMMARY

The sarcoplasmic reticulum (SR) is a sub-cellular organelle responsible for regulating the Ca2+ concentration in the cytosol of muscle fibers (W. HASSELBACH and M. MAKINOSE, ATP and active transport. Biochem Biophys Res Commun 7, 132-136 (1962). By hydrolysis of ATP, the SR network lowers the free Ca2+ concentration in the space surrounding the myofibrils to sub-micromolar levels, pumping Ca2+ into the lumen of the SR. The reduction of myoplasmic free Ca2+ concentration leads to muscle relaxation.

Muscle contraction is initiated by an action potential at the cell's surface membrane. This depolarization propagates down the transverse (T) tubules, which in turn triggers the release of Ca2+ stored in the SR and contraction. More particularly, calcium release channels (CRCs) in the SR called ryanodine receptors (RyRs) open and release Ca2+ from the SR into the intracellular cytoplasm of the cell. Release of Ca2+ into the cytoplasm from the SR increases cytoplasmic Ca2+ concentration. Open probability (Po) of the RyR receptor refers to the likelihood that the RyR channel is open at any given moment, and therefore capable of releasing Ca2+ into the cytoplasm from the SR.

There are three types of ryanodine receptors, all of which are highly-related Ca2+ channels: RyR1, RyR2, and RyR3. RyR1 is found predominantly in skeletal muscle as well as other tissues, while RyR2 is found predominantly in the heart as well as other tissues, and RyR3 is found in the brain as well as other tissues. The RyR channels are formed by four RyR polypeptides in association with four FK506 binding proteins (FKBPs), specifically FKBP12 (calstabinl) and FKBP12.6 (calstabin2). Calstabinl binds to RyR1, calstabin2 binds to RyR2, and calstabinl binds to RyR3. The FKBP proteins (calstabinl and calstabin2) bind to the RyR channel (one molecule per RyR subunit), stabilize RyR-channel functioning, and facilitate coupled gating between neighboring RyR channels, thereby preventing abnormal activation of the channel during a closed state.

Recent advances have been made toward understanding the 3-dimensional structure of the ryanodine receptor (RyR)/Ca2+ release protein, and the possible functional role of other junctional SR proteins in excitation contraction coupling (ECC) in skeletal muscle. As such, ECC differs in skeletal and cardiac muscle. In skeletal muscle, there appears to be a mechanical coupling between the dihydropyridine receptor (DHPR) found in the T-tubule membrane and the CRC or RyR found at the terminal end of the SR(M. F. Schneider and W. K. Chandler, Voltage dependent charge movement of skeletal muscle: a possible step in excitation-contraction coupling. Nature 242, 244-246 (1973)). On the other hand, in cardiac muscle, Ca2+ enters the cell during the action potential through the DHPR, and initiates Ca2+ release from the SR via a mechanism known as Ca2+-induced Ca2+ release (A. Fabiato, Calcium-induced release of calcium from the cardiac sarcoplasmic reticulum. Am J Physiol 245, C1-14 (1983).

A number of associated proteins regulate the activity of the SR ryanodine receptors. The DHPR and RyR appear to form a hub for a large macromolecular complex, which includes triadin and calsequestrin (on the luminal face of the SR), FKBP12 (skeletal muscle) and FKBP12.6 (cardiac muscle), calmodulin, Ca2+-CaM kinase (skeletal muscle), and protein kinase A (PKA) (cardiac muscle). Defective RyR-FKBP12.6 association has been implicated in heart failure, cardiomyopathy, cardiac hypertrophy, and exercise induced sudden cardiac death. It has been proposed that PKA phosphorylation of the cardiac RyR2 results in dissociation of FKBP12.6 from the Ca2+ release channel, which results in an increased channel open probability (Po), increased sensitivity to activation by Ca2+, and destabilization of the CRC (X. H. Wehrens, S. E. Lehnart, S. R. Reiken, S. X. Deng, J. A. Vest, D. Cervantes, J. Coromilas, D. W. Landry and A. R. Marks, Protection from cardiac arrhythmia through ryanodine receptor-stabilizing protein calstabin2. Science 304, 292-296 (2004). Alternatively, it has been proposed that abnormal Ca2+ handling by calsequestrin may lead to an increased Ca2+ leak and cardiac arrhythmias. The cardio-protective agent K201 (also known as JTV519) and the antioxidant edaravone appear to correct the defective FKBP12.6 control of RyR2 and improve function. However, the mechanism of action of K201 is controversial. One report has shown that K201 suppresses spontaneous Ca2+ release in ventricular myocytes independent of the presence of the FKBP12.6 protein, suggesting that the mode by which K201 decreases the Ca2+ leak from cardiac SR does not involve the FKBP12.6 protein D. J. Hunt, P. P. Jones, R. Wang, W. Chen, J. Bolstad, K. Chen, Y. Shimoni and S. R. Chen, K201 (JTV519) suppresses spontaneous Ca2+ release and [3H]ryanodine binding to RyR2 irrespective of FKBP12.6 association. Biochem J 404, 431-438 (2007).

In addition, CRCs from both cardiac and skeletal muscle SR are rich in thiol groups, and therefore, are strongly regulated by thiol reagents. It has been shown that oxidation of these thiol groups results in increased Ca2+ release rates from SR vesicles, increased open probability of the reconstituted CRC, and increased high affinity ryanodine binding to the SR, while reduction of the disulfide(s) formed results in decreased activity (J. L. Trimm, G. Salama and J. J. Abramson, Sulfhydryl oxidation induces rapid calcium release from sarcoplasmic reticulum vesicles. J Biol Chem 261, 16092-16098 (1986). , (J. J. Abramson, E. Buck, G. Salama, J. E. Casida and I. N. Pessah, Mechanism of anthraquinone-induced calcium release from skeletal muscle sarcoplasmic reticulum. J Biol Chem 263, 18750-18758 (1988).) There are also a large number of non-thiol reagents known to either activate or inhibit RyR1 and/or RyR2. Among those compounds that activate the RyR/CRC are methylxanthines such as caffeine, plant alkaloids such as ryanodine, polyamines such as polylysine, quinone such as doxorubicin, and phenols such as 4-chloro-m-cresol (4-CmC). Among the non-thiol RyR/CRC inhibitors are local anesthetics such as tetracaine and procaine, and the poly-unsaturated fatty acids such as docosahexaenoic acid (DHA). These reagents are physiologically and pharmacologically diverse, and their mode of action was somewhat controversial (B. S. Marinov, R. O. Olojo, R. Xia and J. J. Abramson, Non-thiol reagents regulate ryanodine receptor function by redox interactions that modify reactive thiols. Antioxid Redox Signal 9, 609-621 (2007)).

The inventors herein have recognized that RyR2 plays an important role during excitation-contraction coupling, and further that antiarrhythmic compounds targeting the RyR2 channel complex do not interfere with systolic SR Ca2+ release. At the same time, inhibition of diastolic SR Ca2+ release is a desirable feature of compounds that might prevent arrhythmias. The inventors have addressed this issue by showing that substantially all pharmacological inhibitors of RyR channels are electron donors. And, the inventors have demonstrated that an exchange of electrons is a common molecular mechanism involved in modifying the function of the RyR. The inventors have further developed a redox model wherein an underlying channel modulation of function was supported by observations that inhibitors of the RyR1 shift the thiol/disulfide balance within RyR1 to a more reduced state, while channel activators shift this balance to a more oxidized state (R. Xia, T. Stangler and J. J. Abramson, Skeletal muscle ryanodine receptor is a redox sensor with a well defined redox potential that is sensitive to channel modulators. J Biol Chem 275, 36556-36561 (2000)). Therefore, the molecular mechanism underlying the action of some drugs appears to involve the formation of a charge-transfer complex linking the added drug and RyR, which results in a shift in the redox status of reactive thiols. The inventors have developed a novel assay based on the redox model to quantify the ability of drugs to either donate or accept electrons, and have demonstrated a strong correlation between the potency of RyR inhibitors and their effectiveness to act as electron donors (B. S. Marinov, R. O. Olojo, R. Xia and J. J. Abramson, Non-thiol reagents regulate ryanodine receptor function by redox interactions that modify reactive thiols Antioxid Redox Signal 9, 609-621 (2007).

Thus, by generating derivatives of known RyR inhibitors that have enhanced electron donor properties, drugs having high potency as RyR inhibitors can be generated. By then assaying the derivatives for in vivo and in vitro efficacy, toxicity, and selectivity, novel anti-arrhythmia drugs may be developed.

In one particular example, the inventors have synthesized drugs having enhanced electron donor properties that target RyR2 while being highly effective in decreasing the SR Ca2+ leak associated with ventricular arrhythmias. For example, studies carried out on RyR1 demonstrate that synthesizing a 4-methoxy derivative of 4-chloro-3-methyl phenol (4-CmC) converts an electron acceptor/channel activator into an electron donor/channel inhibitor. The newly synthesized compound, 4-methoxy-m-cresol (4-MmC) is thus a strong electron donor, and a potent inhibitor of both RyR1 and RyR2. The inventors further found that the electron donor properties correlate well with the drug's effectiveness as a channel inhibitor, although the three dimensional structure of drug binding sites to each of the proteins are currently unknown, which makes computer assisted drug design difficult in practice. Thus, by using the electron donor properties of compounds as a basis for identifying new RyR1/RyR2 targeting drugs, drug discovery can be performed without requiring extensive structure based or computer assisted drug design (Yanping Ye, D. Y., Laura J. Owen, Jorge O. Escobedo, Jialu Wang, Jeffrey D. Singer, Robert M. Strongin and Jonathan J. Abramson, Designing Calcium Release Channel Inhibitors with Enhanced Electron Donor Properties: Stabilizing the Closed State of Ryanodine Receptor Type 1. Molecular Pharmacology, 2012. 81: p. 53-62).

The inventors have developed a strategy to create new drugs with enhanced electron donor properties to target RyR2 that are highly effective in decreasing the SR Ca2+ leak associated with ventricular arrhythmias. As such, stronger electron donors are more potent inhibitors of the Ca2+ leak which enables their use at lower effective concentrations and introduces the possibility of decreasing harmful side-effects associated with commonly used drugs that target ventricular arrhythmias. This non-traditional approach toward designing drugs to target RyR2 (or other proteins) contrasts the rapid screening technology in general use. Preliminary results have shown that the developed approach is successful in designing new more potent anti-arrhythmogenic compounds which are orders of magnitude more effective than presently exist.

In light of the foregoing, it is possible to provide novel compounds and/or methods for regulating or modulating the activity of calcium release channels such as ryanodine receptors, in cells of a subject (e.g., mammals, preferably humans), thereby overcoming various deficiencies and shortcomings known in the field. In particular, because abnormal Ca2+ release through RyR2 has emerged as a substantial mechanism of arrhythmogenesis, the inventors herein show that a lead compound that targets RyR2, referred to herein as Compound 1, also suppresses arrhythmias. Thus, in the normal heart, Ca2+ release from the SR via RyR2 is a tightly regulated process that involves discrete release of Ca2+ during systole, and cessation of Ca2+ release during diastole. For the timely rhythmic release of Ca2+ from RyR2, the channel must open in response to a cytoplasmic Ca2+ flux, but remain closed during diastolic SR Ca2+ filling. Destabilization of RyR2 may occur as a result of genetic mutations (e.g., Catecholaminergic Polymorphic Ventricular Tachycardia, or CPVT) or acquired modifications (e.g., oxidation, nitrosylation, phosphorylation). The common consequence of both genetic and acquired modifications in RyR2 is an increased propensity towards pathologic SR Ca2+ release during diastole, which can initiate cardiac arrhythmias.

In addition, it can be possible to provide novel compounds and/or methods for inhibiting or decreasing intracellular calcium release, including calcium release in muscle cells (e.g., from SR in skeletal or cardiac muscle cells). These compounds and/or methods can include down-regulating or inhibiting the activity of calcium release channels such as ryanodine receptors.

Further, it may be possible to provide compounds and/or methods for changing the redox potential of reactive thiols on ryanodine receptors in cells of a subject. Such redox potential changes can be achieved by modifying the thiol/disulfide balance within ryanodine receptors in cells of a subject, particularly, mammalian cells (R. Xia, T. Stangler and J. J. Abramson, Skeletal muscle ryanodine receptor is a redox sensor with a well defined redox potential that is sensitive to channel modulators. J Biol Chem 275, 36556-36561 (2000)).

Further still, it may be possible to provide compounds and/or methods for treating or reducing the risk of a ryanodine receptor (RyR) associated disease, disorder, or condition in a subject. In particular, the RyR-associated disorder, disease, or condition can be a cardiac or skeletal muscle condition, disorder, or disease. For example, the compounds according to the present disclosure may be used to treat CPVT arrhythmias, (e.g., by targeting one or more of RyR1, RyR2, and RyR3), or ventricular arrhythmias, atrial arrhythmias, heart failure, skeletal muscle fatigue, and cardiac disease linked to diabetes, and hypertension.

Further still, herein, the lead compound Compound 1 is characterized in a mouse model of Catecholaminergic Polymorphic Ventricular Tachycardia (or CPVT), for example. CPVT is an orphan disease that affects approximately 1/10,000 people (e.g., humans). The condition is a severe genetic arrhythmogenic disorder characterized by adrenergically induced ventricular tachycardia (VT) that manifests as syncope and sudden death. As one example, a typical age of CPVT onset is between 7 and 9 years of age for both male and female genders. Syncopal spells, brought on by exercise or acute emotion, are frequently the first symptom observed, although sudden death can be the first manifestation of the disease for a subset of patients (10-20%). The three genes linked to CPVT are the cardiac ryanodine receptor (RyR2) gene, which is the cause of CPVT in approximately 55% to 65% of cases, and the cardiac calsequestrin (CAS Q2) and triadin genes. Such genetic defects are associated with a disruption of normal Ca2+ homeostasis in affected individuals (Pott, C., et al., Successful treatment of catecholaminergic polymorphic ventricular tachycardia with flecainide: a case report and review of the current literature. Europace. 13(6): p. 897-901).

Accordingly, in part, the present teachings provide Compounds 1-5 (shown below in Table 1), and pharmaceutically acceptable formulations and prodrugs thereof. The present teachings also provide methods of associated conditions, disorders, and diseases comprising administering a therapeutically effective amount of Compounds 1-5 to a subject in need thereof. In addition, the present teachings relate to methods of reducing the open probability of a ryanodine receptor, and methods of reducing Ca2+ release across a ryanodine receptor (e.g., into the cytoplasm of a cell), either of which can include contacting Compounds 1-5 with a ryanodine receptor.

The above advantages and other advantages, and features of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The advantages described herein will be more fully understood by reading an example of an embodiment, referred to herein as the Detailed Description, when taken alone or with reference to the drawings, where:

FIG. 1 shows an inhibitory effect of the compound Compound 1 on the spark frequency of cells derived from a CPVT mouse model;

FIGS. 2-3 depict the effect of the compound Compound 1 on arrhythmias at a whole animal level in CPVT mice;

FIG. 4 shows a confocal line-scan image of Ca2+ spark recordings from isolated ventricular myocytes; and

FIGS. 5 A and B show exemplary derivatives of Tetracaine.

### DETAILED DESCRIPTION

Throughout the application, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” or the symbol “˜” refers to a ±10% variation from the nominal value unless otherwise indicated or inferred.

It should be understood that the order of steps or order for performing certain actions is immaterial so long as the present teachings remain operable. Moreover, two or more steps or actions may be conducted simultaneously.

As used herein, a “compound” refers to the compound itself and its pharmaceutically acceptable salts, hydrates, complexes, esters, prodrugs and/or salts of prodrugs, unless otherwise understood from the context of the description or expressly limited to one particular form of the compound, that is, the compound itself, or a pharmaceutically acceptable salt, hydrate, complex, ester, prodrug or salt of prodrug thereof.

As used herein, “halo” or “halogen” refers to fluoro, chloro, bromo, and iodo.

As used herein, “alkyl” refers to a straight-chain or branched saturated hydrocarbon group. Examples of alkyl groups include methyl (Me), ethyl (Et), propyl (e.g., n-propyl and iso-propyl), butyl (e.g., n-butyl, iso-butyl, sec-butyl, tert-butyl), pentyl groups (e.g., n-pentyl, iso-pentyl, neopentyl), hexyl groups, and the like. In various embodiments, an alkyl group can have 1 to 40 carbon atoms (e.g., C1-40 alkyl group), for example, 1-20 carbon atoms (e.g., C1-20 alkyl group). In some embodiments, an alkyl group can have 1 to 6 carbon atoms, and can be referred to as a “lower alkyl group.” Examples of lower alkyl groups include methyl, ethyl, propyl (e.g., n-propyl and iso-propyl), and butyl groups (e.g., n-butyl, iso-butyl, sec-butyl, tert-butyl). In some embodiments, alkyl groups can be substituted as described herein.

As used herein, “alkoxy” refers to —O-alkyl group. Examples of alkoxy groups include methoxy, ethoxy, propoxy (e.g., n-propoxy and isopropoxy), t-butoxy, pentoxyl, hexoxyl groups, and the like.

As used herein, “alkylthio” refers to an —S-alkyl group (which, in some cases, can be expressed as —S(O)w-alkyl, wherein w is 0). Examples of alkylthio groups include methylthio, ethylthio, propylthio (e.g., n-propylthio and isopropylthio), t-butylthio, pentylthio, hexylthio groups, and the like.

As used herein, “cycloalkyl” refers to a non-aromatic carbocyclic group including cyclized alkyl, alkenyl, and alkynyl groups. In various embodiments, a cycloalkyl group can have 3 to 24 carbon atoms, for example, 3 to 20 carbon atoms (e.g., C3-14 cycloalkyl group). A cycloalkyl group can be monocyclic (e.g., cyclohexyl) or polycyclic (e.g., containing fused, bridged, and/or spiro ring systems), where the carbon atoms are located inside or outside of the ring system. Any suitable ring position of the cycloalkyl group can be covalently linked to the defined chemical structure. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcaryl, adamantyl, and spiro[4.5]decanyl groups, as well as their homologs, isomers, and the like. In some embodiments, cycloalkyl groups can be substituted as described herein.

As used herein, “heteroatom” refers to an atom of any element other than carbon or hydrogen and includes, for example, nitrogen, oxygen, silicon, sulfur, phosphorus, and selenium.

As used herein, “cycloheteroalkyl” refers to a non-aromatic cycloalkyl group that contains at least one ring heteroatom selected from O, S, Se, N, P, and Si (e.g., O, S, and N), and optionally contains one or more double or triple bonds. A cycloheteroalkyl group can have 3 to 24 ring atoms, for example, 3 to 20 ring atoms (e.g., 3-14 membered cycloheteroalkyl group). One or more N, P, S, or Se atoms (e.g., N or S) in a cycloheteroalkyl ring may be oxidized (e.g., morpholine N-oxide, thiomorpholine S-oxide, thiomorpholine S,S-dioxide). In some embodiments, nitrogen or phosphorus atoms of cycloheteroalkyl groups can bear a substituent, for example, a hydrogen atom, an alkyl group, or other substituents as described herein. Cycloheteroalkyl groups can also contain one or more oxo groups, such as oxopiperidyl, oxooxazolidyl, dioxo-(1H,3H)-pyrimidyl, oxo-2(1H)-pyridyl, and the like. Examples of cycloheteroalkyl groups include, among others, morpholinyl, thiomorpholinyl, pyranyl, imidazolidinyl, imidazolinyl, oxazolidinyl, pyrazolidinyl, pyrazolinyl, pyrrolidinyl, pyrrolinyl, tetrahydrofuranyl, tetrahydrothiophenyl, piperidinyl, piperazinyl, and the like. In some embodiments, cycloheteroalkyl groups can be substituted as described herein.

As used herein, “aryl” refers to an aromatic monocyclic hydrocarbon ring system or a polycyclic ring system in which two or more aromatic hydrocarbon rings are fused (e.g., having a bond in common with) together or at least one aromatic monocyclic hydrocarbon ring is fused to one or more cycloalkyl and/or cycloheteroalkyl rings. An aryl group can have 6 to 24 carbon atoms in its ring system (e.g., C6-20 aryl group), which can include multiple fused rings. In some embodiments, a polycyclic aryl group can have 8 to 24 carbon atoms. Any suitable ring position of the aryl group can be covalently linked to the defined chemical structure. Examples of aryl groups having only aromatic carbocyclic ring(s) include phenyl, 1-naphthyl (bicyclic), 2-naphthyl (bicyclic), anthracenyl (tricyclic), phenanthrenyl (tricyclic), pentacenyl (pentacyclic), and like groups. Examples of polycyclic ring systems in which at least one aromatic carbocyclic ring is fused to one or more cycloalkyl and/or cycloheteroalkyl rings include, among others, benzo derivatives of cyclopentane (e.g., an indanyl group, which is a 5,6-bicyclic cycloalkyl/aromatic ring system), cyclohexane (e.g., a tetrahydronaphthyl group, which is a 6,6-bicyclic cycloalkyl/aromatic ring system), imidazoline (e.g., a benzimidazolinyl group, which is a 5,6-bicyclic cycloheteroalkyl/aromatic ring system), and pyran (e.g., a chromenyl group, which is a 6,6-bicyclic cycloheteroalkyl/aromatic ring system). Other examples of aryl groups include benzodioxanyl, benzodioxolyl, chromanyl, indolinyl groups, and the like. In some embodiments, aryl groups can be substituted as described herein. In some embodiments, an aryl group can have one or more halogen substituents, and can be referred to as a “haloaryl” group. Perhaloaryl groups, that is, aryl groups where all of the hydrogen atoms are replaced with halogen atoms (e.g., —C6F5), are included within the definition of “haloaryl.” In certain embodiments, an aryl group is substituted with another aryl group and can be referred to as a biaryl group. Each of the aryl groups in the biaryl group can be substituted as disclosed herein.

As used herein, “heteroaryl” refers to an aromatic monocyclic ring system containing at least one ring heteroatom selected from oxygen (O), nitrogen (N), sulfur (S), silicon (Si), and selenium (Se) or a polycyclic ring system where at least one of the rings present in the ring system is aromatic and contains at least one ring heteroatom. Polycyclic heteroaryl groups include those having two or more heteroaryl rings fused together, as well as those having at least one monocyclic heteroaryl ring fused to one or more aromatic carbocyclic rings, non-aromatic carbocyclic rings, and/or non-aromatic cycloheteroalkyl rings. A heteroaryl group, as a whole, /can have, for example, 5 to 24 ring atoms and contain 1-5 ring heteroatoms (e.g., 5-20 membered heteroaryl group). The heteroaryl group can be attached to the defined chemical structure at any heteroatom or carbon atom that results in a stable structure. Generally, heteroaryl rings do not contain O—O, S—S, or S—O bonds. However, one or more N or S atoms in a heteroaryl group can be oxidized (e.g., pyridine N-oxide, thiophene S-oxide, thiophene S,S-dioxide). Examples of heteroaryl groups include, for example, the 5- or 6-membered monocyclic and 5-6 bicyclic ring systems shown below:

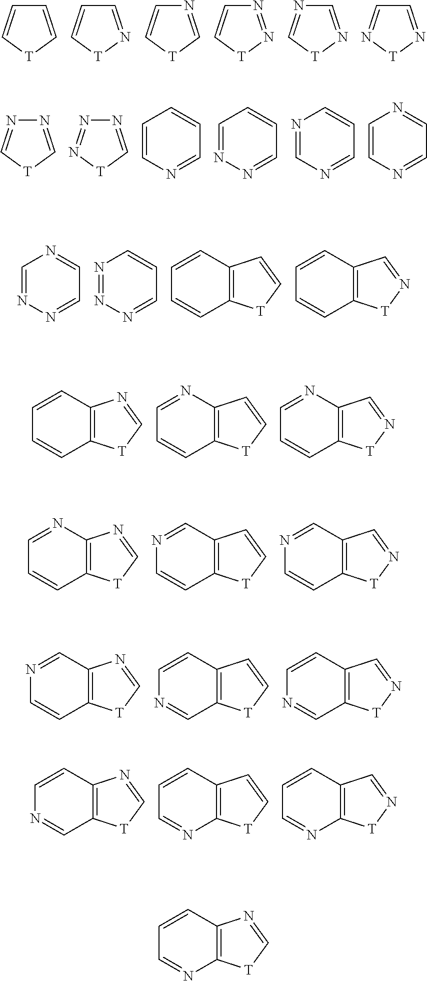

where T is O, S, NH, N-alkyl,N-(arylalkyl) (e.g., N-benzyl), SiH₇, SiH(alkyl), Si(alkyl)₂, SiH(arylalkyl), Si(arylalkyl)₂, or Si(alkyl)(arylalkyl). Examples of such heteroaryl rings include pyrrolyl, furyl, thienyl, pyrimidyl, pyr dazinyl, pyrazinyl, triazolyl, tetrazlyl, pyrazolyl, imidazolyl, isothiazolyl, thiazolyl, thiadiazolyl, isoxazolyl, oxazolyl, oxadiazolyl, indolyl, isoindolyl, benzofuryl, benzothienyl, quinolyl, 2-ethylquinolyl, isoquinolyl, quinoxalyl, quinazolyl, benzotriarolyl, benzimidazolyl, benzothiazolyl, benzisothiazolyl, benzisoxazolyl, be zoxadiazolyl, benzoxazolyl, cinnolinyl, 1H-indazolyl, 2H-indazolyl, indolizinyl, isobenzofuyl, naphthyridinyl, phthalazinyl, pteridinyl, purinyl, oxazolopyridinyl, thiazolopyridinyl, imidazopyridinyl, furopyridinyl, thienopyridinyl, pyridopyrimidinyl, pyridopyraziyl, pyridop thienothiazolyl, thienoxazolyl, thienoirnidazolyl groups, and the like. Further examples of heteroaryl groups include 4,5,6,7-tetrahydroindolyl, tetrahyciroquinolinyl, benzothienopyridinyl, benzofuropyridinyl groups, and the like. in some embodiments, heteroaryl groups can be substituted as described herein.

Compounds of the present teachings can include a “divalent group” defined herein as a linking group capable of forming a covalent bond with two other moieties. For example, compounds of the present teachings can include a divalent C1-20 alkyl group (e.g., a methylene group), a divalent C2-20 alkenyl group (e.g., a vinylyl group), a divalent C2-20 alkynyl group (e.g., an ethynylyl group), a divalent C6-14 aryl group (e.g., a phenylyl group), a divalent 3-14 membered cycloheteroalkyl group (e.g., a pyrrolidylyl), and/or a divalent 5-14 membered heteroaryl group (e.g., a thienylyl group). Generally, a chemical group (e.g., —Ar—) is understood to be divalent by the inclusion of the two bonds before and after the group.

The electron-donating or electron-withdrawing properties of several hundred of the most common substituents, reflecting all common classes of substituents have been determined, quantified, and published. Quantification of electron-donating and electron-withdrawing reference, which lists Hammett σ values for properties may be expressed in terms of Hammett σ values. Hydrogen has a Hammett σ value of zero, while other substituents have Hammett σ values that increase positively or negatively in direct relation to their electron-withdrawing or electron-donating characteristics. Substituents with negative Hammett σ values are considered electron-donating, while those with positive Hammett σ values are considered electron-withdrawing. For example, see Lange's Handbook of Chemistry, 12th ed., McGraw Hill, 1979, Table 3-12, pp. 3-134 to 3-138, incorporated herein by a large number of commonly encountered substituents.

It should be understood that the term “electron-accepting group” can be used synonymously herein with “electron acceptor” and “electron-withdrawing group”. In particular, an “electron-withdrawing group” (“EWG”) or an “electron-accepting group” or an “electron-acceptor” refers to a functional group that is electrophilic and draws electrons to itself more than a hydrogen atom would if it occupied the same position in a molecule. Electron-withdrawing groups can be conjugated or not conjugated with the core molecule. Examples of electron-withdrawing groups include —NO2, —CN, —NC, halogen or halo (e.g., F, Cl, Br, I), —S(R0)2 +, —(R0)3 +, —SO3H, —SO2R0, —SO3R0, —SO2NHR0, —SO2N(R0)2, —COOH, COR0, —COOR0, —CONHR0, —CON(R0)2, C1-40 haloalkyl groups, C6-14 aryl groups, and 5-14 membered electron-poor heteroaryl groups; where R0 is a C1-20 alkyl group, a C2-20 alkenyl group, a C2-20 alkynyl group, a C 1-20 haloalkyl group, a C 1-20 alkoxy group, a C6-14 aryl group, a C3-14 cycloalkyl group, a 3-14 membered cycloheteroalkyl group, and a 5-14 membered heteroaryl group, each of which can be optionally substituted as described herein. For example, each of the C1-20 alkyl group, the C2-20 alkenyl group, the C2-20 alkynyl group, the C1-20 haloalkyl group, the C1-20 alkoxy group, the C6-14 aryl group, the C3-14 cycloalkyl group, the 3-14 membered cycloheteroalkyl group, and the 5-14 membered heteroaryl group can be optionally substituted with 1-5 small electron-withdrawing groups such as F, Cl, Br, —NO2, —CN, —NC, —S(R0)2 +, —N(R0)3 +, —SO3H, —SO2R0, —SO3R0, —SO2NHR0, —SO2N(R0)2, —COOH, —COR0, —COOR0, —CONHR0, and —CON(R0)2.

It should be understood that the term “electron-donating group” can be used synonymously herein with “electron donor”. In particular, an “electron-donating group” or an “electron-donor” refers to a functional group that donates electrons to a neighboring atom more than a hydrogen atom would if it occupied the same position in a molecule. Examples of electron-donating groups include chalcogen-containing groups such as —OH, —OR0, —SH, SR0, and selenides, where R0 is as defined above. Other electron-donating groups include nitrogen-containing groups such as optionally substituted amino groups (—NH2, —NHR0, N(R0)2) and hydrazines, and 5-14 membered electron-rich heteroaryl groups. Other examples of electron-donating groups include electropositive groups which may work through non-resonance effects. Examples of such electropositive groups include silyl groups. Still, additional examples of electron-donating groups include saturated and unsaturated groups such as alkyl groups, alkenyl groups, aryl groups, and alkynyl groups which can increase electron-donating properties via both resonance and non-resonance effects and which can be optionally substituted with 1-4 groups independently selected from —OH, —OR0, —SH, —SR0, —NH2, —NHR0, —N(R0)2, and 5-14 membered electron-rich heteroaryl groups, where R0 is as defined above.

Various unsubstituted heteroaryl groups can be described as electron-rich (or π-excessive) or electron-poor (or π-deficient). Such classification is based on the average electron density on each ring atom as compared to that of a carbon atom in benzene. Examples of electron-rich systems include 5-membered heteroaryl groups having one heteroatom such as furan, pyrrole, and thiophene; and their benzofused counterparts such as benzofuran, benzopyrrole, and benzothiophene. Examples of electron-poor systems include 6-membered heteroaryl groups having one or more heteroatoms such as pyridine, pyrazine, pyridazine, and pyrimidine; as well as their benzofused counterparts such as quinoline, isoquinoline, quinoxaline, cinnoline, phthalazine, naphthyridine, quinazoline, phenanthridine, acridine, and purine. Mixed heteroaromatic rings can belong to either class depending on the type, number, and position of the one or more heteroatom(s) in the ring. For example, see Katritzky, A. R and Lagowski, J. M., Heterocyclic Chemistry (John Wiley & Sons, New York, 1960), incorporated herein by reference.

At various places in the present specification, substituents are disclosed in groups or in ranges. It is specifically intended that the description include each and every individual subcombination of the members of such groups and ranges. For example, the term “C1-6 alkyl” is specifically intended to individually disclose Cl, C2, C3, C4, C5, C6, C1-C6, C1-05, C1-C4, C1-C3, C1-C2, C2-C6, C2-C5, C2-C4, C2-C3, C3-C6, C3-C5, C3-C4, C4-C6, C4-05, and C5-C6 alkyl. By way of other examples, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20. Additional examples include that the phrase “optionally substituted with 1-5 substituents” is specifically intended to individually disclose a chemical group that can include 0, 1, 2, 3, 4, 5, 0-5, 0-4, 0-3, 0-2, 0-1, 1-5, 1-4, 1-3, 1-2, 2-5, 2-4, 2-3, 3-5, 3-4, and 4-5 substituents.

Compounds described herein can contain an asymmetric atom (also referred as a chiral center) and some of the compounds can contain two or more asymmetric atoms or centers, which can thus give rise to optical isomers (enantiomers) and diastereomers (geometric isomers). The present teachings include such optical isomers and diastereomers, including their respective resolved enantiomerically or diastereomerically pure isomers (e.g., (+) or (−) stereoisomer) and their racemic mixtures, as well as other mixtures of the enantiomers and diastereomers. In some embodiments, optical isomers can be obtained in enantiomerically enriched or pure form by standard procedures known to those skilled in the art, which include, for example, chiral separation, diastereomeric salt formation, kinetic resolution, and asymmetric synthesis. For example, when a compound of the present teachings is a racemate, the racemate can be separated into the (S)-compound and (R)-compound by optical resolution. The present teachings also encompass cis- and trans-isomers of compounds containing alkenyl moieties (e.g., alkenes, azo, and imines). It also should be understood that the compounds of the present teachings encompass all possible regioisomers in pure form and mixtures thereof. In some embodiments, the preparation of the present compounds can include separating such isomers using standard separation procedures known to those skilled in the art, for example, by using one or more of column chromatography, thin-layer chromatography, simulated moving-bed chromatography, and high-performance liquid chromatography. However, mixtures of regioisomers can be used similarly to the uses of each individual regioisomer of the present teachings as described herein and/or known by a skilled artisan.

It is specifically contemplated that the depiction of one regioisomer includes any other regioisomers and any regioisomeric mixtures unless specifically stated otherwise.

As used herein, a “leaving group” (“LG”) refers to a charged or uncharged atom (or group of atoms) that can be displaced as a stable species as a result of, for example, a substitution or elimination reaction. Examples of leaving groups include halogen (e.g., Cl, Br, I), azide (N3), thiocyanate (SCN), nitro (NO2), cyanate (CN), water (H2O), ammonia (NH3), and sulfonate groups (e.g., OSO2—R, wherein R can be a C1-10 alkyl group or a C6-14 aryl group each optionally substituted with 1-4 groups independently selected from a C1-10 alkyl group and an electron-withdrawing group) such as tosylate (toluenesulfonate, OTs), mesylate (methanesulfonate, OMs), brosylate (p-bromobenzenesulfonate, OB s), nosylate (4-nitrobenzenesulfonate, ONs), and triflate (trifluoromethanesulfonate, OTf).

Throughout the specification, structures may or may not be presented with chemical names. Where any question arises as to nomenclature, the structure prevails.

In one aspect, the present teachings provide derivatives of tetracaine having the formula:

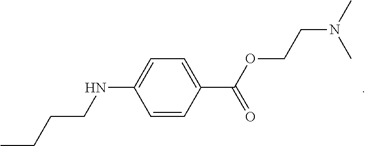 [SMILES: CCCCNc1ccc(C(=O)OCCN(C)C)cc1]

Thus, tetracaine is referred to as the parent molecule and derivatives formed according to the present teachings include one or more-electron donating groups added into the base structure above. That is, the present derivatives generally have enhanced electron-donating properties compared to the parent molecule tetracaine. The inventors have found that such derivatives can act as Ca2+ release channel (CRC) inhibitors, and that their potency can be as high as ˜2500 times that of tetracaine.

More specifically, the present teachings provide derivative compounds of tetracaine having the following structures:

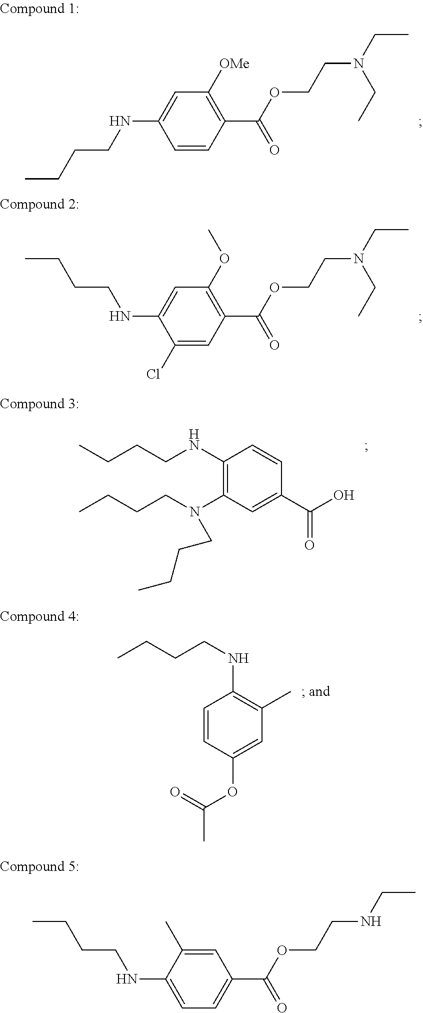

Studies have shown that lead Compound 1 inhibits calcium spark frequency in cells derived from a CPVT mouse model with an 1050=35 nM (TABLE 1) while also decreasing arrhythmias in the whole animal at a level of 2.5 [symbol] g/kg. For this reason, according to the methods disclosed, Compound 1, which is a di-ethyl amine derivative of tetracaine, may be further included within a pharmaceutical composition administered to a subject with cardiac arrhythmia to suppress or reduce the arrhythmia. Because tetracaine derivatives according to the present methods target ryanodine receptors to reduce sarcoplasmic reticulum Ca2+ leaks, in some instances, advantages are realized by delivering the drug in a neutral, or non-salt, form of the compound. Charged ions may not interactact as strongly with the ryanodine receptor, which may reduce a drug potency compared to a compound with a non-salt form. For this reason, the compound may be included within the pharmaceutical composition in a non-salt form based on an expected potency for inhibition relative to the parent molecule. When the compound in the non-salt form is included within the pharmaceutical composition administered to the subject with cardiac arrhythmia, the compound may be included therein in a therapeutically effective amount.

As one example, methods for reducing a cardiac arrhythmia in a subject using a di-ethyl amine derivative of tetracaine are enabled according to the present disclosure. Thus, Compound 1 with the formula:

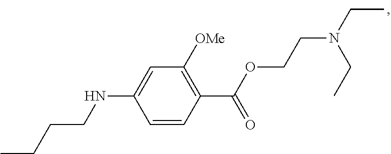 [SMILES: CCCCNc1ccc(C(=O)OCCN(CC)CC)c(OC)c1]

may be used for reducing the cardiac arrhythmia in the subject, wherein reducing the cardiac arrhythmia in the subject includes administering a therapeutically effective amount of the di-ethyl amine derivative of tetracaine. Furthermore, methods are enabled wherein the di-ethyl amine derivative of tetracaine is included in the therapeutically effective amount within a pharmaceutical composition administered to the subject with cardiac arrhythmia. As noted above, because the di-ethyl amine derivative of tetracaine includes electron rich groups, the potency for inhibition of RyR2 is increased compared to the parent molecule, as indicated by the studies herein described. To reduce charge-charge interactions with the ryanodine receptor, which in some instances may reduce binding of the drug to the receptor, the methods further include adding the di-ethyl amine derivative of tetracaine within the pharmaceutical composition in a non-salt form in some instances to increase receptor binding, and thereby the potency of the drug. Although the experimental data described below relate to studies in a mouse population, the methods may be applied to subject that are mammals, in general, and in particular may be applied to human subjects for reducing a cardiac arrhythmia.

In this way, method are enabled, that comprise administering to a mammal suffering from cardiac arrhythmia, a therapeutically effective amount of a compound, the compound selected from the group consisting of:

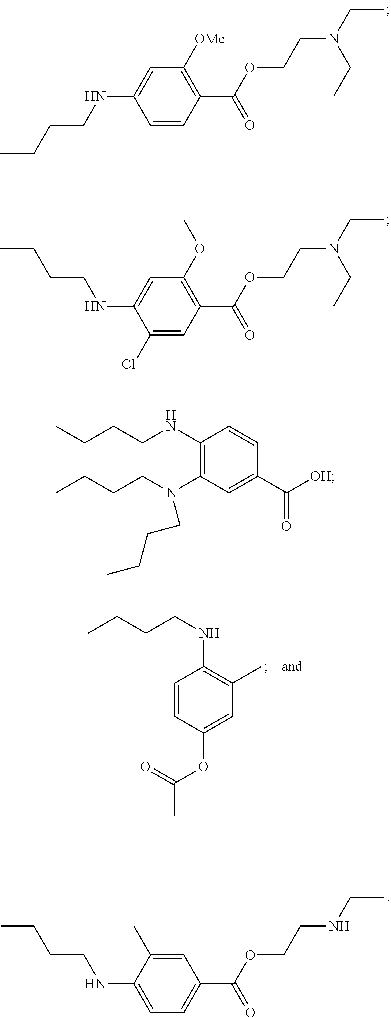

Although not described explicitly herein, in some instances, advantages may arise wherein more than one of the compounds indicated as Compound 1-5 are included in a pharmaceutical composition. In this way, the methods may also include combinations thereof.

TABLE 1 shows potency and toxicity data for a select group of compounds developed according to the methods described, and for which dose response curves have been measured. For example, Compound 1 (Entry 1 of TABLE 1) and analogs (entries 3-6 of TABLE 1) are more electron rich and potent than the parent structure tetracaine (Entry 2 of TABLE 1).

TABLE 1

| | | | | Cytotoxicity (IC₅₀, | |
|---|---|---|---|---|---|
| | | | IC₅₀ | μM) | |
| Entry | Compound | Structure | (nM) | HEK293 | HepG2 |
| 1 | Compound 1 | 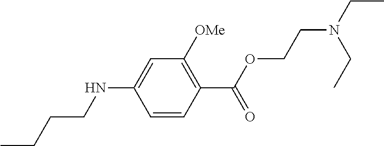 [SMILES: CCCCNc1ccc(C(=O)OCCN(CC)CC)c(OC)c1] | 35 | Nontoxic at 50 μM | Nontoxic at 50 μM |
| 2 | Tetracaine | 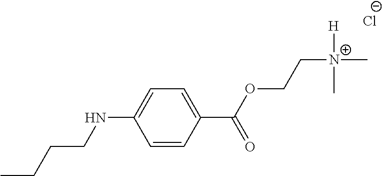 [SMILES: [Cl-] ; CCCCNc1ccc(C(=O)OCC[NH+](C)C)cc1] | 60 000- 100 000 | Nontoxic at 50 μM | Nontoxic at 50 μM |
| 3 | Compound 2 | 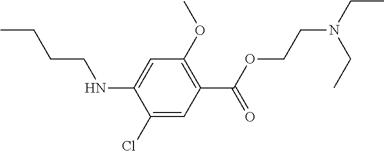 [SMILES: CCCCNc1cc(OC)c(C(=O)OCCN(CC)CC)cc1Cl] | 11 | Nontoxic at 50 μM | Nontoxic at 50 μM |
| 4 | Compound 3 | 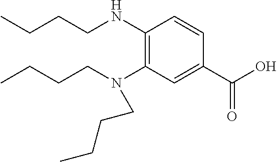 [SMILES: CCCCNc1ccc(C(=O)O)cc1N(CCCC)CCCC] | 500 | Nontoxic at 50 μM | Nontoxic at 50 μM |
| 5 | Compound 4 | 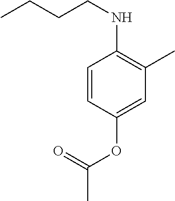 [SMILES: CCCCNc1ccc(OC(C)=O)cc1C] | 630 | 6.7 μM | 47 μM |
| 6 | Compound 5 | 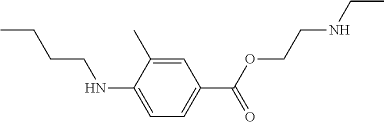 [SMILES: CCCCNc1ccc(C(=O)OCCNCC)cc1C] | 800 | Nontoxic at 50 μM | Nontoxic at 50 μM |

The present teachings provide for the compounds of Table 1 to be used for reducing cardiac arrhythmia. For example, a compound for reducing cardiac arrhythmia with the formula:

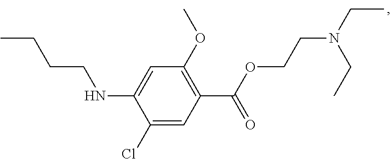 [SMILES: CCCCNc1cc(OC)c(C(=O)OCCN(CC)CC)cc1Cl]

that is, Compound 2, may be used to reduce cardiac arrhythmia in a subject. In the way, methods are enabled wherein the compound is included in a therapeutically effective amount within a pharmaceutical composition administered to a subject having cardiac arrhythmia.

Likewise, a compound for reducing cardiac arrhythmia with the formula:

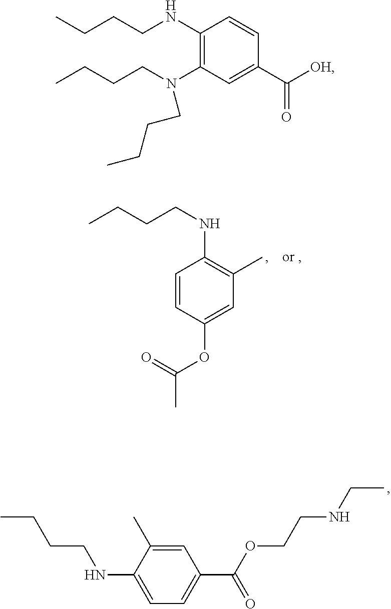

that is, Compounds 3, 4, or 5 may alternatively or additionally be used to reduce cardiac arrhythmia in a subject. In the way, methods are enabled wherein the compound is included in a therapeutically effective amount within a pharmaceutical composition administered to a subject having cardiac arrhythmia.

More generally, the present teachings provide compounds (e.g., derivatives of tetracaine or tetracaine analogs) of Formula I:

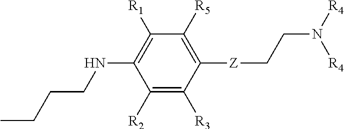

tetracaine analogs:
R₁, R₂, R₃=combinations of
H, alkyl, O—R, OH, NHR, NRR, halide and R4=H, alkyl or a combination of H and alkyl
R5=H or OR
Z=ester carbonyl and oxygen or amide or urea
   wherein R¹, R², and R³ independently are selected from H, O-alkyl, OH, NH-alkyl, N,N-dalkyl, and halo fluoro, chloro, bromo, and iodo), R4 is alkyl, H or a combination of H and alkyl; R5 is H or 0-alkyl; and Z is an ester carbonyl group, amide or urea. Thus, tetracaine derivatives may be formed from combinations of these groups formed in the base structure shown by formula I. For example, some of Compounds 1-5 (Table 1) are derivatives of Formula I.

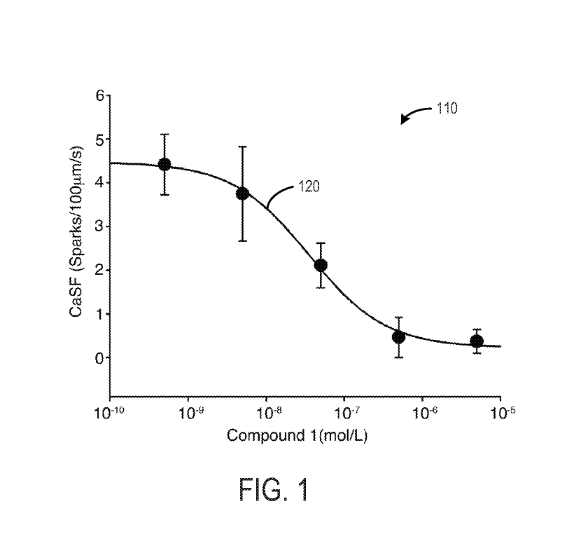

Turning now to the action of the compounds in animal studies according to the present disclosure, FIG. 1 shows an inhibitory effect via data plot 110 of the compound Compound 1 on the spark frequency of cells derived from a CPVT mouse model. Therein, calcium spark frequency data are shown in the presence of increasing amounts of added compound. IC50 is a measure of the effectiveness of a substance in inhibiting a particular biological process or function. Calcium spark frequency is plotted along the y-axis and compound concentration is plotted along the x-axis. Example dose-response curve 120 is included to illustrate the inhibitory effect of Compound 1 on spark frequency for the cells derived from a CPVT mouse model. The quantitative value of IC50 can be determined by identifying the concentration where half of the maximum biological response, such as the calcium spark response, is inhibited. For example, at lower compound concentrations dose-response curve 110 has a higher level of calcium spark frequency, whereas at higher compound concentrations, a transition of the calcium spark frequency to a lower level is observed. The concentration at which the sigmoidal curve of data response curve 120 has a spark frequency reduced by half corresponds to the IC50. As such, Compound 1 inhibits spark frequency in cells derived from a CPVT mouse model with an IC50=35 nM (e.g., 3.5 e-8 M), and decreases arrhythmias in the whole animal level at 2.5 μg/kg as described in greater deteail below. Thus, as one example, a method for treating cardiac arrhythmias may include administering an amount of Compound 1 to a subject afflicted with heart arrhythmias. The administration may be performed via known routes, such as oral, epidermal, subcutaneous, intravenous, peritoneal, etc.

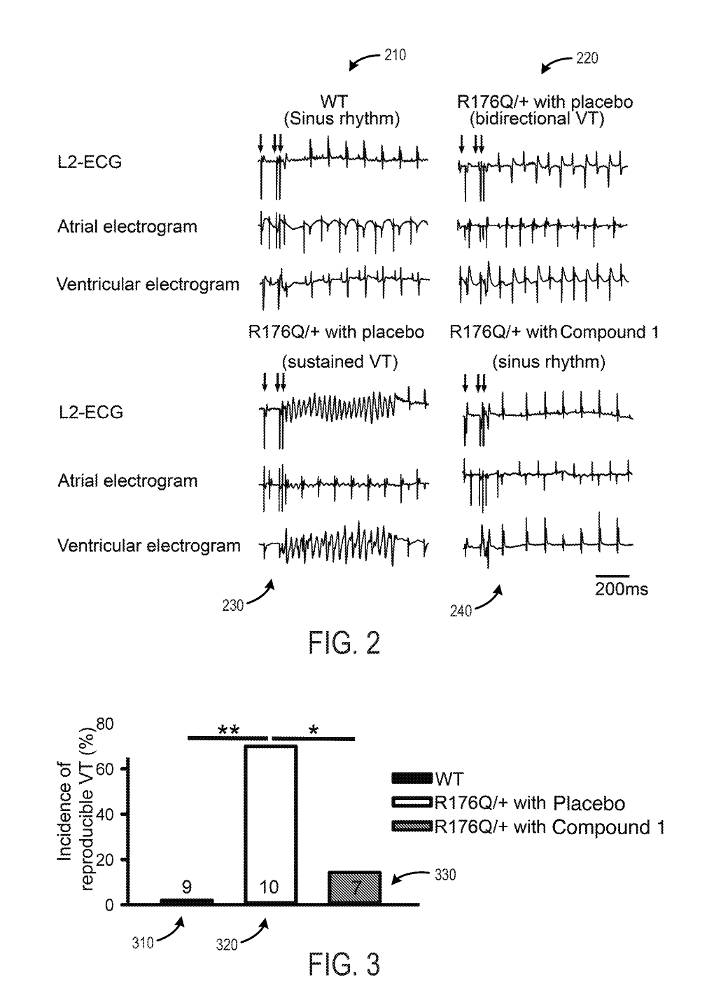

FIGS. 2-3 further depict the effect of Compound 1 on arrhythmias in CPVT mice at a whole animal level. Ventricular tachycardia (or VT) is a type of tachycardia, or a rapid heart beat that arises from improper electrical activity of the heart that presents as a rapid heart rhythm. VT is associated with the bottom chambers of the heart, called the ventricles, which are pumping chambers of the heart. FIG. 2 shows example electrical activity data to illustrate the effect of the drug for treating cardiac arrhythmias using two different arrhythmogenic models. Therein, electrical signals for four different CPVT mice are shown. Wild type (or WT) signal 210 is provided for reference to illustrate a sinus rhythm (e.g., L2-ECG, lead 2 of the surface ECG). Atrial and ventricular intracardiac electrocardiogram signals are also provided for reference. Placebo-treated R176/+ mice developed bidirectional VT (signal 220) or sustained polymorphic VT (signal 230) following cardiac pacing (arrows). In contrast, R176Q/+ mice treated with lead compound Compound 1 were protected from arrhythmic development following pacing, and exhibited normal sinus rhythm (signal 240).

FIG. 3 shows a histogram of the incidence of reproducible VT for each of the mouse models just described. WT histogram 310 shows that the incidence of VT for a wild type mouse is low relative to the R176Q/+ mice following programmed electrical stimulation. For example, placebo histogram 320 illustrates a higher rate of VT incidence compared to WT histogram 310. In addition, drug histogram 330 illustrates a lower incidence of VT than placebo histogram 320, which according to the present disclosure may further indicate an increased drug efficacy.

The advantage of the methods according to the present disclosure is that increased inhibition and drug efficacy (e.g., a drug ˜3000 times more potent than the parent molecule from which it was derived) may be administered at a lower dosage. For example, the concentration of lead compound Compound 1 that suppressed ventricular tachycardia is approximately 0.5% of compound K201 (JTV519) that was previously used to inhibit arrhythmias in a similar mouse model. Further, lead compound Compound 1, when injected in the tail of the mouse was shown to inhibit arrhythmias within 10 minutes. In contrast, K201 required subcutaneous implantation administered over a period of three days and lengthened the Q-T interval (Wehrens, X.H., et al., Enhancing calstabin binding to ryanodine receptors improves cardiac and skeletal muscle function in heart failure. Proc Natl Acad Sci USA, 2005. 102(27): p. 9607-12). Therefore, advantages exist with regard to reduction of dosage response time compared to previously used drugs to inhibit arrhythmias. No indication has been observed that Compound 1 has any effect on the length of the AP. While Compound 1 inhibits sparks and arrhythmias at 35 nM, the ATPase activity of SERCA2 has been measured versus concentration of Compound 1. At concentrations of 1 μM, 10 μM and 50 μM, ATPase activity was unaffected (data not shown). The data shown in FIGS. 2 and 3 have been repeated several times with different animals (e.g., rabbit model).

In view of the above, the inventors have identified strategies for developing new drugs aimed at decreasing the Ca2+ leak from cardiac SR based on increasing electron donor properties of preexisting drugs, which represents a new approach to drug development. To demonstrate the effectiveness of this approach, the inventors have developed exemplary drugs to treat ventricular arrhythmias using derivatives of the RyR2 inhibitor tetracaine. As noted above, Compound 1 is ˜3000 times more effective than tetracaine at inhibiting RyR2 activity. Moreover, it shows antiarrhythmic activity in the CPVT mouse model at a concentration which is 0.1% of the pharmaceutical flecainide (Watanabe, H., et al., Flecainide prevents catecholaminergic polymorphic ventricular tachycardia in mice and humans. Nat Med, 2009. 15(4): p. 380-3). For comparison, most previous inhibitors of the Ca2+ leak from SR showed a relatively low potency and poor specificity.

The inventors have further examined the specificity of newly developed compounds, and given the enhanced potency (-3-4 orders of magnitude) have found that it is likely that newly developed drugs may also be more specific at targeting the RyR2 Ca2+ leak.

Methods to study newly synthesized compounds include:

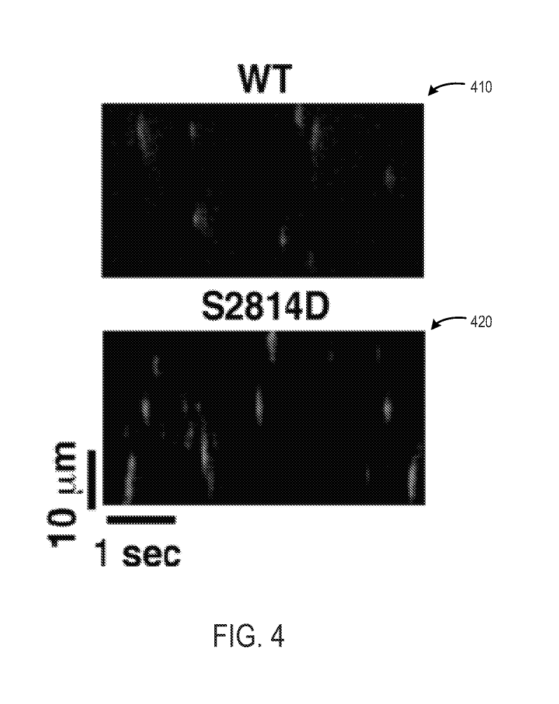

Functional Assays in CPVT mouse cells may include isolating ventricular cardiomyocytes from adult (2-3 month-old) R176Q/+ mice or WT littermates. In one exemplary assay, myocytes were loaded with 2 μM Fluo-4-AM in a normal Tyrode solution containing 1.8 mM Ca2+ for 30 minutes at room temperature before washing the cells with Tyrode solution for 15 minutes for de-esterification and transfering to a chamber equipped with parallel platinum electrodes. FIG. 4 shows fluorescence images recorded in line-scan mode with 1024 pixels per line at 500 Hz using a LSM510 confocal microscope for the isolated ventricular cardiomyocytes indicated (e.g., WT and S2814D). Once steady state Ca2+ transiently induced by 1 Hz-pacing (5 ms, 10 V) was observed, pacing was stopped and Ca2+ sparks were monitored for 45 seconds. The cells were then exposed to 100 nM isoproterenol (ISO) to induce an increase in spontaneous Ca2+ spark activity associated with arrhythmogenesis (Kannankeril, P.J., et al., Mice with the R176Q cardiac ryanodine receptor mutation exhibit catecholamine-induced ventricular tachycardia and cardiomyopathy. Proc Natl Acad Sci USA, 2006. 103(32): p. 12179-84.).

After baseline measurements were completed, myocytes were exposed to the test lead compound. After a 5 min superfusion period, Ca2+ sparks were again measured in the same cell in the presence of the drug compound. The frequency of Ca2+ sparks (CaSpF) were calculated, e.g., using SparkMaster (Picht, E., et al., SparkMaster: automated calcium spark analysis with ImageJ. Am J Physiol Cell Physiol, 2007. 293(3): p. C1073-81), and applications before and after inclusion of the test compound were compared. A detailed dose-response relationship and measurements of inhibition of arrhythmogenic Ca2+ waves in ventricular myocytes from a CPVT mouse model (R176Q/+) were then completed on those compounds showing a significant decrease in spark frequency at 0.5 μM. At the conclusion of these experiments, the Ca2+ spark inhibition rate is calculated as a function of concentration for each compound tested, and an IC50 value determined for each drug compound. As shown in FIG. 4, a gain-of-function mutation in RyR2 (referred to as S2814D) leads to an increased incidence of spontaneous Ca2+ sparks. FIG. 4 compares an exemplary confocal line-scan image of Ca2+ spark recordings from ventricular myocytes isolated from wild-type (WT) mice at 410 relative to those isolated from S2814D mice as shown at 420.

With regard to the design and synthesis of more potent RyR2 inhibitors, the inventors herein describe an approach based on enhancing the electron donor properties of existing compounds. For this reason, the methods described may include assessing the potency of the drugs as inhibitors of RyR2 activity at each of a molecular level and a cellular level. In some instances, this includes determining a potency in normalizing Ca2+ homeostasis and decreasing arrhythmias at the one or more of the cellular and whole animal level.

Ca2+ leak through RyR2 has emerged as a mechanism of arrhythmogenesis. As such, Ca2+ release via RyR2 is a highly regulated process involving the discrete release of Ca2+ during systole, and termination of Ca2+ release during diastole. Genetic mutations, such as CPVT, as well as acquired modifications (such as oxidation, nitrosylation, phosphorylation, etc.) result in the destabilization of RyR2, which may result in increased pathologic release of Ca2+ during diastole, and thereby initiate cardiac arrhythmias (Marx, S. O., et al., PKA phosphorylation dissociates FKBP12.6 from the calcium release channel (ryanodine receptor): defective regulation in failing hearts. Cell, 2000. 101(4): p. 365-76., Chelu, M. G., et al., Calmodulin kinase II-mediated sarcoplasmic reticulum Ca2+ leak promotes atrial fibrillation in mice. J Clin Invest, 2009. 119(7): p. 1940-51., van Oort, R. J., et al., Ryanodine receptor phosphorylation by calcium/calmodulin-dependent protein kinase II promotes life-threatening ventricular arrhythmias in mice with heart failure. Circulation, 2010. 122(25): p. 2669-79).

Exemplary drugs known to target RyR2 include benzothiazepine and its derivatives and flecainide. These drugs reduce the open probability of RyR2, and thereby reduce the pathologic Ca2+ leak. Since RyR2 plays a role in excitation-contraction coupling, anti-arrhythmic compounds targeting the RyR2 channel complex may be designed to inhibit diastolic Ca2+ release, while not interfering with systolic Ca2+ release. Since pharmacological inhibitors of RyR channels (including RyR1, RyR2 and RyR3 channels) tend to be electron donors, it follows that an exchange of electrons may lie at the core of the molecular function. In this way, the redox model underlying channel modification, and further involving the formation of a charge-transfer complex, is supported by observations that inhibitors of RyR1 shift the thiol/disulfide balance within RyR to a more reduced state, while channel activators shift the balance towards a more oxidized state. According to the present disclosure, this property can be advantageously used to design compounds that target and modify RyR channels while providing anti-arrhythmic activity (B. S. Marinov, R. O. Olojo, R. Xia and J. J. Abramson, Non-thiol reagents regulate ryanodine receptor function by redox interactions that modify reactive thiols Antioxid Redox Signal 9, 609-621 (2007).

Results of studies described above performed at the cellular level indicate that inherited mutations in RyR2, identified in patients suffering from catecholamine polymorphic ventricular tachycardia (CPVT), cause an increased susceptibility towards exercise or catecholamine-induced polymorphic ventricular arrhythmias Likewise, mice heterozygous for mutation R176Q in RyR2 are more vulnerable to VT following catecholamine stimulation, with R176Q/+ mice exhibiting an increased incidence of isoproterenol-induced, spontaneous Ca2+ release events.

The above-discussed Ca2+ spark assay was used with R176Q/+ myocytes as an initial screening assay to test the ability of new compounds to inhibit spontaneous pathological SR Ca2+ release in ventricular myocytes.

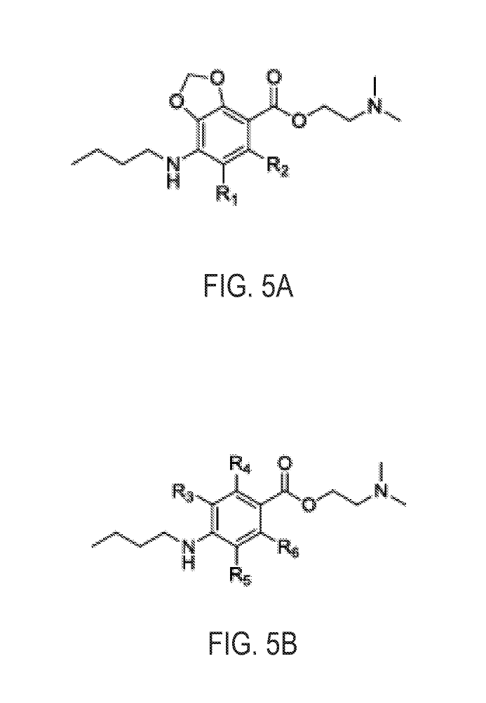

As described herein, modification of known RyR2 inhibitors to generate derivatives having increased electron donor properties offer attractive potential for new drugs with increased inhibition of ventricular tachycardia. FIGS. 5 A and B show exemplary tetracaine analogs functionalized with electron donating groups based on analogous syntheses. Tetracaine is a local anesthetic and also a Na+ channel inhibitor. Tetracaine has been shown to inhibit the RyR at ˜150 μM, but can lead to SR Ca2+ overload in cardiac myocytes and spontaneous Ca2+ release from SR (Xu, L., R. Jones, and G. Meissner, Effects of local anesthetics on single channel behavior of skeletal muscle calcium release channel. J. Gen. Physiol, 1993. 101(2): p. 207-233).

One feature of the known pharmaceuticals JTV519 (K201), Flecainide, Tetracaine, Ranolazine, and Verapamil is an apparent lack of specificity. The inventors have recognized the potency of these drugs can be increased substantially to inhibit the SR Ca2+ leak by increasing their electron donor properties via addition of chemical moieties that increase the electron donor properties. The inventors have further recognized that the newly generated drugs with increased electron donor properties may advantageously decrease non-specific effects while also targeting RyR2 at lower concentrations, which may result in further synergistic benefits.

TABLES 2 and 3 show exemplary tetracaine analogs functionalized with electron donating groups. In particular, TABLE 2 shows example electron donating groups and corresponding arrangements within the tetracaine derivative indicated. Likewise, TABLE 3 shows example electron donating groups and corresponding arrangements within the tetracaine derivative molecular structure indicated. In this way, additional compounds may be represented according to the present disclosure by incorporating the R-group indicated for each entry into the tetracaine derivative structure provided at the identified location to arrive at an electron rich compound.

TABLE 2

| | 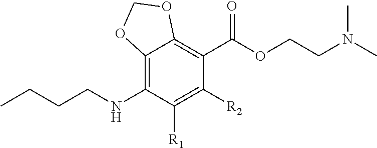 | |
|---|---|---|
| Entry | R1 | R2 |
| 1 | O-alkyl | H |
| 2 | O-alkyl | O-alkyl |
| 3 | H | O-alkyl |
| 4 | H | H |
| 5 | N(alkyl)₂ | H |
| 6 | N(alkyl)₂ | N(alkyl)₂ |
| 7 | H | N(alkyl)₂ |
| 8 | OH | H |
| 9 | H | OH |
| 10 | N(alkyl)₂ | O-alkyl |
| 11 | O-alkyl | N(alkyl)₂ |

TABLE 3

| | 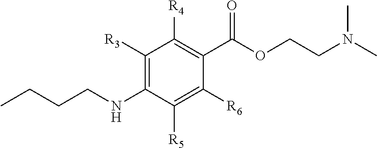 | | | |
|---|---|---|---|---|
| Entry | R3 | R4 | R5 | R6 |
| 1 | H | OH | H | H |
| 2 | H | OH | H | OH |
| 3 | H | O-alkyl | H | N(alkyl)₂ |
| 4 | O-alkyl | H | H | N(alkyl)₂ |
| 5 | N(alkyl)₂ | H | H | O-alkyl |
| 6 | O-alkyl | N(alkyl)₂ | N(alkyl)₂ | H |
| 7 | O-alkyl | N(alkyl)₂ | H | N(alkyl)₂ |
| 8 | O-alkyl | H | N(alkyl)₂ | N(alkyl)₂ |
| 9 | N(alkyl)₂ | O-alkyl | N(alkyl)₂ | H |
| 10 | N(alkyl)₂ | O-alkyl | H | N(alkyl)₂ |
| 11 | H | O-alkyl | N(alkyl)₂ | N(alkyl)₂ |
| 12 | N(alkyl)₂ | O-alkyl | O-alkyl | H |
| 13 | N(alkyl)₂ | O-alkyl | H | O-alkyl |
| 14 | N(alkyl)₂ | H | O-alkyl | O-alkyl |
| 15 | O-alkyl | N(alkyl)₂ | O-alkyl | H |
| 16 | O-alkyl | N(alkyl)₂ | H | O-alkyl |
| 17 | H | N(alkyl)₂ | O-alkyl | O-alkyl |
| 18 | O-alkyl | N(alkyl)₂ | N(alkyl)₂ | N(alkyl)₂ |
| 19 | N(alkyl)₂ | O-alkyl | N(alkyl)₂ | N(alkyl)₂ |
| 20 | N(alkyl)₂ | O-alkyl | O-alkyl | O-alkyl |
| 21 | O-alkyl | N(alkyl)₂ | O-alkyl | O-alkyl |
| 22 | O-alkyl | H | H | H |
| 23 | H | O-alkyl | H | H |
| 24 | O-alkyl | O-alkyl | H | H |
| 25 | O-alkyl | H | O-alkyl | H |
| 26 | H | O-alkyl | H | O-alkyl |
| 27 | O-alkyl | H | H | O-alkyl |
| 28 | O-alkyl | O-alkyl | O-alkyl | H |
| 29 | O-alkyl | O-alkyl | H | O-alkyl |
| 30 | N(alkyl)₂ | H | H | H |
| 31 | H | N(alkyl)₂ | H | H |
| 32 | N(alkyl)₂ | N(alkyl)₂ | H | H |
| 33 | N(alkyl)₂ | H | N(alkyl)₂ | H |
| 34 | H | N(alkyl)₂ | H | N(alkyl)₂ |
| 35 | N(alkyl)₂ | H | H | N(alkyl)₂ |
| 36 | N(alkyl)₂ | N(alkyl)₂ | N(alkyl)₂ | H |
| 37 | N(alkyl)₂ | N(alkyl)₂ | H | N(alkyl)₂ |
| 38 | O-alkyl | N(alkyl)₂ | H | H |
| 39 | N(alkyl)₂ | O-alkyl | H | H |
| 40 | O-alkyl | H | N(alkyl)₂ | H |
| 41 | OH | H | H | H |
| 42 | OH | H | OH | H |

It will be appreciated that while the example drugs discussed herein are assessed with reference to their effect on CPVT, this is non-limiting. In alternate examples, the novel compounds generated and described may additionally or alternatively be used to address one or more RyR-associated disorders, diseases, or conditions including cardiac or skeletal muscle conditions, disorders, or diseases. For example, the compounds may be used to reduce the risk of CPVT arrhythmias, (e.g., by targeting one or more of RyR1, RyR2, and RyR3). As another example, the compounds may be used to reduce the risk of ventricular arrhythmias, atrial arrhythmias (such as atrial fibrillation, atrial flutter, etc.), diastolic heart failure, heart failure with reduced ejection fraction, pregnancy-induced cardiomyopathy, hypertrophic cardiomyopathy, dilated cardiomyopathy, skeletal muscle fatigue, and cardiac disease linked to diabetes, and hypertension. In still further examples, the compounds may be used to reduce the risk of malignant hyperthermia, central core disease, heart-stroke, myopathy, diabetes (e.g., diabetic cardiomyopathy, etc.), Duchenne muscular dystrophy, Becker muscular dystrophy, aging-related cognitive dysfunction, chronic obstructive pulmonary disease (COPD), bladder dysfunction, and incontinence. The present disclosure also allows for the use of the substances disclosed in the manufacture of a medicament for the treatment the conditions just described. For example, Compound 1 may be used in the manufacture of a medicament for the treatment of cardiac arrhythmia. In another example, Compound 1 may be used for the treatment of ventricular arrhythmia.

In this way, novel compounds/drugs can be generated from known RyR inhibitors by adding derivatives that enhance the electron donor properties of the compound. By using the electron donor properties of the compounds to preliminarily assess their inhibitor potency, a large number of compounds can be rapidly and reliably tested. This allows for the development of new more potent anti-arrhythmic drugs, which can be used at lower concentrations while showing less non-specific effects.

1. A compound having the formula: 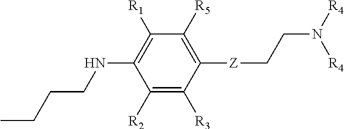
wherein R1, R2, and R3 are independently selected from the group consisting of H, alkyl, O-alkyl, OH, NH-alkyl, N,N-dialkyl and halide;
R4 is one of H, alkyl and a combination thereof;
R5 is one of H or O-alkyl, and
Z is an ester carbonyl group.

2. The compound of claim 1, further included within a pharmaceutical composition administered to a subject with cardiac arrhythmia.

3. The compound of claim 1, wherein the compound is included within the pharmaceutical composition in therapeutically effective amount.

4. The compound of claim 1, wherein the compound included in the pharmaceutical composition in the therapeutically effective amount in a non-salt.

5. The compound of claim 1, having the formula:

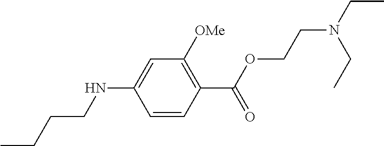 [SMILES: CCCCNc1ccc(C(=O)OCCN(CC)CC)c(OC)c1]

6. The compound of claim 1, having the formula:

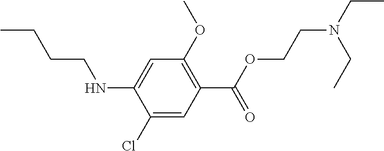 [SMILES: CCCCNc1cc(OC)c(C(=O)OCCN(CC)CC)cc1Cl]

7. The compound of claim 1, having the formula:

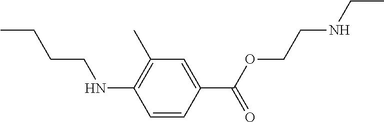 [SMILES: CCCCNc1ccc(C(=O)OCCNCC)cc1C]

8. A compound with the formula:

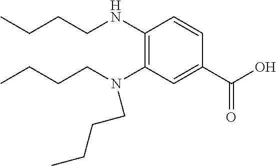 [SMILES: CCCCNc1ccc(C(=O)O)cc1N(CCCC)CCCC]

9. The compound of claim 8, wherein the compound is included in a therapeutically effective amount within a pharmaceutical composition administered to a subject with cardiac arrhythmia.

10. A compound with the formula:

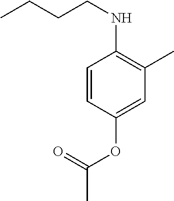 [SMILES: CCCCNc1ccc(OC(C)=O)cc1C]

11. The compound of claim 10, wherein the compound is included in a therapeutically effective amount within a pharmaceutical composition administered to a subject with cardiac arrhythmia.

12. A non-salt compound with the formula:

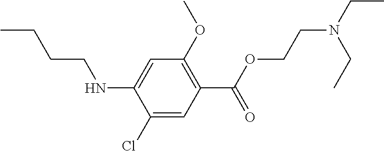 [SMILES: CCCCNc1cc(OC)c(C(=O)OCCN(CC)CC)cc1Cl]

13. The non-salt compound of claim 12, wherein the compound is included in a therapeutically effective amount within a pharmaceutical composition administered to a subject with cardiac arrhythmia.

14. A method for reducing a cardiac arrhythmia in a subject using a derivative of tetracaine with the formula: 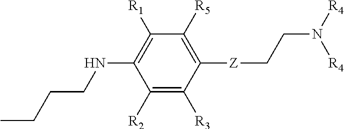
wherein R1, R2, and R3 are independently selected from the group consisting of H, alkyl, O-alkyl, OH, NH-alkyl, N,N-dialkyl and halide;
R4 is one of H, alkyl, and a combination thereof;
R5 is one of H or 0-alkyl; and
Z is an ester carbonyl group.

15. The method of claim 14, further including administering a therapeutically effective amount of the derivative of tetracaine to the subject with cardiac arrhythmia.

16. The method of claim 14, wherein the derivative of tetracaine in the therapeutically effective amount is included within a pharmaceutical composition administered to the subject with cardiac arrhythmia.

17. The method of claim 14, wherein the derivative of tetracaine is included within the pharmaceutical composition in a non-salt form.

18. The method of claim 14, wherein the subject is a human.

19. The method of claim 14, wherein the derivative of tetracaine has the formula

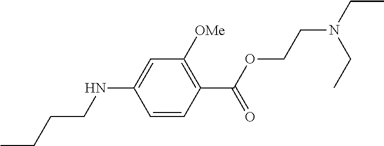 [SMILES: CCCCNc1ccc(C(=O)OCCN(CC)CC)c(OC)c1]

20. The method of claim 14, wherein the derivative of tetracaine is one of

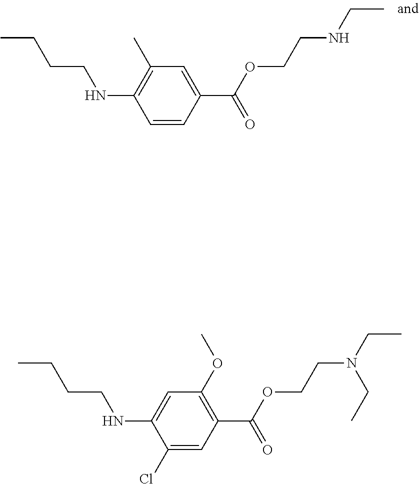

21. A method comprising:
administering to a mammal suffering from cardiac arrhythmia, a therapeutically effective amount of a compound, the compound selected from the group consisting of: [chemical structure]
